(19) United States
(12) Patent Application Publication
(10) Pub. No.: US 2003/0108510 A1
(43) Pub. Date: Jun. 12, 2003

(54) Skin cream stimulating the surface bloodstream

(76) Inventor: Siro Passi, Rome (IT)

Correspondence Address: YOUNG & THOMPSON, 745 SOUTH 23RD STREET 2ND FLOOR, ARLINGTON, VA, 22202

(21) Appl. No.: 10/182,129

(22) PCT Filed: Nov. 22, 2001
(86) PCT No.: PCT/IT01/00588

(30) Foreign Application Priority Data
Nov. 29, 2000 (IT) RM20000A000627

(51) Int. Cl. A61K 31/74

(52) U.S. Cl. 424/78.03

(57) ABSTRACT

A formulation of a skin cream based on the synergical combination of three particular active principles—escin, bufemin and rutin—whose pharmacological action stimulating the venous and arterial branchings of the peripheral bloodstream and the perivasal tissues is further increased both by the presence of antioxidants and inhibitors of histidine decarboxylase and the high concentration in soya lecithin (3%) and sebum-similar oil (2-10 %) causing the active principles to penetrate the skin where the three active principles should perform their action. Said cream finds therapeutical application in the treatment of cellulitis and disorders connected to the inefficiency of the surface bloodstream, phlebopathy, phlebothrombosis, surface periphlebitis, sense of heaviness, fatigue and weariness of limbs, spasms, tumefaction and edema of inflammatory origin, ecchymosis and hematoma, and chilblains.

The present invention relates to cosmetics and more particularly a formulation for topical administering to stimulate the surface bloodstream and to eliminate disorders connected to its inefficiency, particularly the formation of cellulitis.

The disclosed formulation is based upon the synergical combination of three particular active principles, i.e. escin, bufenin, and rutin, whose pharmacological action stimulating the venous and arterial branchings of the peripheral bloodstream and the perivasal tissues is further increased both by the presence of antioxidants and inhibitors of histidine decarboxylase and high concentration in soya lecithin (3%) and sebum-similar oil (2-10%) causing the active principles to penetrate the skin where the three active principles should perform their action.

Bufenin is a peripheral vasodilator acting by a β-adrenergic stimulation and having a direct action to arteriae and arteriolae of the skin and skeletal muscles. Therefore, it is particularly active in the treatment of peripheral vascular diseases.

Escin is proposed as anti-edema agent to treat symptoms connected to insufficiency of venous and lymphatic surface vases in order to produce increased resistance and decreased permeability. Is also shows anti-inflammatory, anti-exudative properties.

Rutin is a flavonoid provided with venotonic, vasculo-protective properties as it increases the resistance of vases and decreases their permeability. It is a strong inhibitor of histidine decarboxylase, elastase, hyaluronidase, and lipoxigenase. In addition, because of its polyphenolic nature it is a strong scavenger of radicals and reactive species of oxygen which are hypergenerated, particularly in the skin, both by uncontrolled processes of venostasis, hypoxemia-ischemia-hyperoxia, inflammation, etc., and oxidative attacks to the environment (radiations, smog, etc.).

In order to help the penetration into the skin, beside soya lecithin a sebum-similar oil is used according to the invention which is the object of a preceding Patent Application of the same Applicant filed in Italy under the No. RM2000A000440. It consists of a mixture of sebum-similar lipids of strictly natural origin which is close to the lipid composition of the human sebum which is the normal product of oil glands. A preferred formulation of such oil mixture (which will thereafter be referred to as oil A*) is as follows:

| | (%) |
|---|---|
| Macadamia oil | 50 |
| Jojoba oil (wax) | 24.5 |
| Avocado oil | 10 |
| Unsaponifiable olive oil (squalene) | 12 |
| Free cholesterol | 1.5 |
| Oleate cholesterol | 0.5 |
| Soya phospholipids | 1.4 |
| BHT (butylidroxytoluene) | 0.1 |

As for the antioxidants, two natural physiological antioxidants of the human sebum are used such as vitamin E (d-RRR-α-tocopherol) (0.5-1.5%) and ubiquinone (CoQ₁₀) (0.02-0.2%) which operates in syntony and synergy to protect sebum from oxidation induced by solar radiation and other oxidizing agents of the environment.

The further element characterizing the formulations of the present Patent Application is the presence of anti-inflammatory agents, free radicals and inhibitors of the histidine decarboxylation.

It is known that histamine, a molecule directly involved in the allergic reaction, is formed from the decarboxylation of histidine.

According to the description of the Patent Application No. RM 2000A000520 of the same Applicant, magnesium lactate (0.2-0.8%) and glycol salicylate (0.5-2.0%) are used as inhibitors of the histidine decarboxylation. Glycol salicylate is also known in the literature as dermal counterirritant and anti-inflammatory.

The antihistaminic action of such molecules was proved experimentally to be synergical with the above-mentioned antioxidants and anti-inflammatory agents such as rutin, the extract of ginkgo biloba (0.1-0.5%), and aloe gel (0.1-0.5%) which is rich in polysaccharides.

According to such teachings, a preferred formulation of a cream stimulating the surface bloodstream according to the present invention will be given herebelow only by way of a not limiting example. Both the preferred proportion and the proportion range allowing the purposes of the present invention to be reached are mentioned for each component.

### EXAMPLE 1

CREAM STIMULATING THE SURFACE BLOODSTREAM

| Ingredients (g, ml) | % | range % |
|---|---|---|
| ACTIVE PRINCIPLES | | |
| Sebum-similar oil (A*) | 5 | 2-10 |
| Escin | 0.5 | 0.2-1.0 |
| Bufenin | 0.5 | 0.2-1.0 |
| Rutin | 0.4 | 0.1-0.6 |
| Quercetin | 0.1 | 0.1-0.6 |
| Magnesium ascorbyl phosphate | 0.4 | 0.2-0.8 |
| Extract of (dry) ginkgo biloba | 0.2 | 0.1-0.5 |
| Vitamin E nicotinate | 0.6 | 0.2-1.5 |
| Ubiquinone | 0.05 | 0.02-0.1 |
| Aloe gel 200:1 | 0.2 | 0.1-0.5 |
| Glycol salicylate | 0.5 | 0.2-1.0 |
| Magnesium lactate | 0.3 | 0.1-0.5 |
| EXCIPIENT | | |
| Soya lecithin (granules) | 3 | |
| Glycerin | 3 | |
| BHT | 0.1 | |
| Microcombin | 0.3 | |
| Light perfume | 0.025 | |
| Imidazolynidil urea (IMU) | 0.1 | |
| Citric acid | 0.2 | |
| Carbopol 934 | 0.9 | |
| EDTA | 0.1 | |
| H₂O | q.s. to 100 | |

It is evident to anyone skilled in the art that both the nature and the quantity of the excipients may vary. It is adjusted with EDTA or lactic acid to a pH 6.

In case of foreign creams, an amount of 1-2% extract of papaya can be added.

It should be noted that instead of bufenin a higher concentration in vitamin nicotinate can be used as peripheral vasodilator.

The vasoactive properties of the active principles relative to the venous, arterial, lymphatic surface vases in association with the action of antioxidant and scavenger of radicals and reactive species of oxygen both of the same active principles and the other specific molecules of the formulation have brought to the following dermotherapeutic indications for the formulation of the present invention, after having tested its clinic effectiveness:

reactivation of the surface bloodstream and relief of the disorders connected to its inefficiency;

lymphedema;

indurative dermo-hypodermitis (cellulitis);

phlebopathy (varices of the lower limbs);

phlebothrombosis and surface periphlebitis;

sense of heaviness, fatigue and weariness of limbs;

night spasms;

tumefaction and edema of inflammatory origin;

ecchymosis and hematoma;

chilblains.

It is evident from the above description that a number of modifications and changes can be made in the formulations without departing from the scope of the invention and that such modifications and changes, including the variations in the nature and composition of the excipients, fall within the protection of the following claims which are intended to cover the general characteristics and specifications of the invention.

1. A formulation of a skin cream for topical use stimulating the surface bloodstream, characterized in that it includes escin, bufenin and rutin as active principles.

2. The formulation of a skin cream as claimed in claim 1, characterized in that the pharmacological action of escin, bufenin and rutin stimulating the venous and arterial branchings of the peripheral bloodstream and the perivasal tissues is further increased both by the presence of antioxidants and inhibitors of histidine decarboxylase and the high concentration in soya lecithin and sebum-similar oil causing the active principles to penetrate the skin where the three active principles should perform their action.

3. The formulation of a skin cream as claimed in claim 2, characterized in that vitamin E and ubiquinone are used as physiological antioxidants in suitable quantities to protect sebum from oxidation induced by solar radiation and other oxidizing agents of the environment.

4. The formulation of a skin cream for topical use as claimed in claims 2 and 3, characterized in that magnesium lactate and glycol salicylate are used as inhibitors of the histidine decarboxylation.

5. The formulation of a skin cream for topical use as claimed in claim 2, characterized in that vitamin E and ubiquinone are used in association with suitable quantities of magnesium ascorbyl phosphate, a stable form of vitamin C.

6. The formulation of a skin cream for topical use as claimed in claim 2, characterized in that there are further provided suitable quantities of ginkgoales of ginkgo biloba as antioxidants.

7. The formulation of a skin cream as claimed in the preceding claims, characterized in that the following active principles are provided in weight proportions of the total weight of the product:

| | % |
|---|---|
| Sebum-similar oil (A*) | 2-10 |
| Escin | 0.2-1.0 |
| Bufenin | 0.2-1.0 |
| Rutin | 0.1-0.6 |
| Quercetin | 0.1-0.6 |
| Magnesium ascorbyl phosphate | 0.2-0.8 |
| Extract of (dry) ginkgo biloba | 0.1-0.5 |
| Vitamin E nicotinate | 0.2-1.5 |
| Ubiquinone | 0.02-0.1 |
| Aloe gel 200:1 | 0.1-0.5 |
| Glycol salicylate | 0.2-1.0 |
| Magnesium lactate | 0.1-0.5 |

8. The formulation of a skin cream as claimed in the preceding claims, characterized in that the formulation is as follows:

| Ingredients (g, ml) | % |
|---|---|
| ACTIVE PRINCIPLES | |
| Sebum-similar oil (A*) | 5 |
| Escin | 0.5 |
| Bufenin | 0.5 |
| Rutin | 0.4 |
| Quercetin | 0.1 |
| Magnesium ascorbyl phosphate | 0.4 |
| Extract of (dry) ginkgo biloba | 0.2 |
| Vitamin E nicotinate | 0.6 |
| Ubiquinone | 0.05 |
| Aloe gel 200:1 | 0.2 |
| Glycol salicylate | 0.5 |
| Magnesium lactate | 0.3 |
| EXCIPIENT | |
| Soya lecithin (granules) | 3 |
| Glycerin | 3 |
| BHT | 0.1 |
| Microcombin | 0.3 |
| Light perfume | 0.025 |
| Imidazolynidil urea (IMU) | 0.1 |
| Citric acid | 0.2 |
| Carbopol 934 | 0.9 |
| EDTA | 0.1 |
| H₂O | q.s. to 100 |

9. The formulation of a skin cream as claimed in the preceding claims, characterized in that it has a therapeutic action in the reactivation of the surface bloodstream and the relief of the disorders connected to its inefficiency, as well as the treatment of lymphedema, indurative dermohypodermitis (cellulitis), phlebopathy, phlebothrombosis and surface periphlebitis, sense of heaviness, fatigue and weariness of limbs, night spasms, tumefaction and edema of inflammatory origin, ecchymosis and hematoma, and chilblains.

10. A formulation of a skin cream for topical use stimulating the surface bloodstream, characterized in that it includes in combination with one another a peripheral dilator, an anti-edema agent, and a vasotonic, vasculo-protecting agent in a suitable quantity for a pharmacological action stimulating the venous and arterial branchings of the peripheral bloodstream and the perivasal tissues.
